(19) United States
(12) Patent Application Publication
(10) Pub. No.: US 2004/0259094 A1
(43) Pub. Date: Dec. 23, 2004

(54) Method of attachment of a biomolecule to a solid surface

(76) Inventors: Raj Odedra, Amersham, Buckinghamshire (GB); Lea Pickering, Amersham, Buckinghamshire (GB)

Correspondence Address: AMERSHAM BIOSCIENCES, PATENT DEPARTMENT, 800 CENTENNIAL AVENUE, PISCATAWAY, NJ, 08855, US

(21) Appl. No.: 10/491,014

(22) PCT Filed: Sep. 26, 2002
(86) PCT No.: PCT/GB02/04369

(30) Foreign Application Priority Data
Sep. 26, 2001 (GB) 0123120.8

(51) Int. Cl. C12Q 1/68; G01N 33/53; C12M 1/34; B05D 3/00

(52) U.S. Cl. 435/6; 435/7.1; 435/287.2; 427/2.11

(57) ABSTRACT

Disclosed is a method for attaching biomolecules to a solid surface and a composition for preparing that surface for attachment. The composition comprises molecules of Formula I and Formula II which are defined as follows:

Y-X-Z-R1 Formula I

Y′-X′-Z′-R2 Formula II

and wherein

R1 is a reactive group which can form a covalent bond with a biomolecule or a group capable of forming a reactive group;

R2 is different to R1 and is present in at least a 10⁴ fold molar excess to R1;

Y and Y′ are groups which can bind to a solid surface;

X and X′ are atoms which are, at least, bivalent; and

Z and Z′ are linker groups.

The present invention relates to the immobilisation of molecules on solid surfaces. In particular, the invention relates to the immobilisation of biomolecules, particularly proteins including polypeptides and nucleic acids, including oligonucleotides and polynucleotides.

Immobilised molecules are typically used in methods for analysis. For example, immobilised polypeptides may be used in immunoassays and ELISA assays whereas immobilised nucleic acids may be used in the study of DNA and RNA and can be used for de novo sequencing, the study of hybridisation events and to compare target nucleic acids.

Recent improvements in the study of nucleic acids have focussed on the development of fabricated arrays of immobilised nucleic acids. These arrays typically consist of a high-density matrix of many polynucleotides (such as templates) immobilised onto distinct ordered areas of a solid support material.

A number of different methods for generating an ordered arrangement of molecules on a solid support have been described. For example, Fodor et al. (Trends in Biotechnology (1994) 12, 19-26) describes ways of assembling nucleic acid arrays using a chemically sensitised glass surface protected by a mask, but exposed at defined areas to allow attachment of suitably modified nucleotides at defined areas. Other methods involve spotting out samples at predetermined sites on a solid support such as a slide by robotic micropipetting techniques (see for example, Schena et al. Science (1995) 270:467-470). Such methods generally result in the attachment of a number of molecules at any one of the predetermined sites.

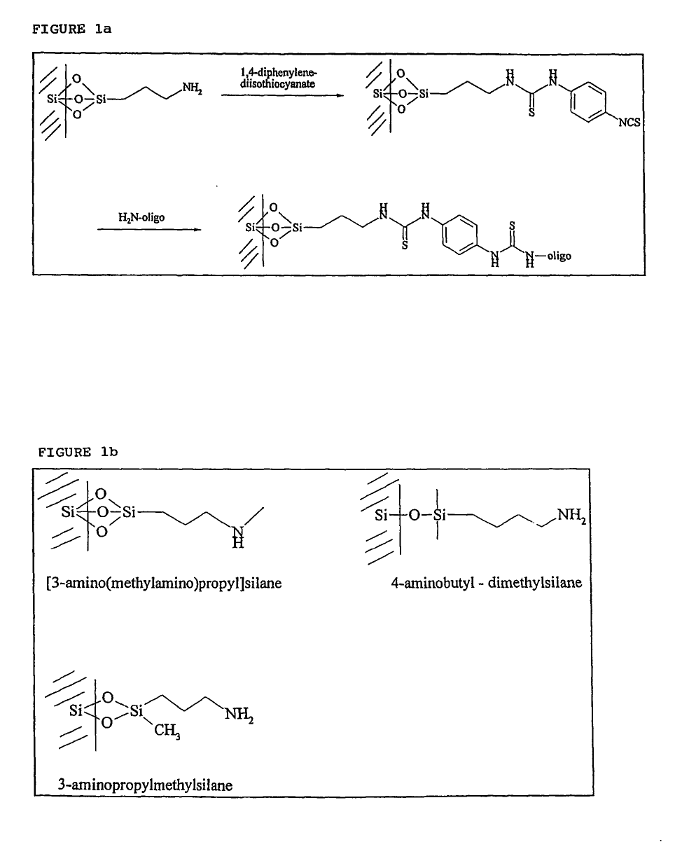

One way of attaching molecules to solid surfaces is to modify the surface by silanisation. For example, U.S. Pat. No. 5,622,826, describes the attachment of 5′-amino modified oligonucleotides, to a glass surface which is first silanised with (3-aminopropyl)triethoxysilane (APTES) to generate a surface containing amino groups. The terminal amino groups on the silanised surface are then reacted with 1,4-diphenylene diisothiocyanate (DPC) to convert the amino groups to phenyleneisothiocyanate groups. These in turn react with the 5′-amino modified oligonucleotides to yield surface bound oligonucleotide. The binding of an oligonucleotide to a surface-bound silane is shown diagrammatically in FIG. 1a. FIG. 1b shows examples of other suitable silanes, bound to surfaces, which may be used to attach biomolecules to solid surfaces.

However, recent advances in methods of single molecule detection (described, for example, in Nie and Zare, Ann. Rev. Biophys. Biomol. Struct., 26, 567-96, 1997) make it possible to detect events such as individual oligonucleotide pairing (Trabesinger, W., et al., Anal Chem., 1999. 71(1); p. 279-83). Tracking events at the single molecule level overcomes some of the problems associated with, for example, previous nucleic acid sequencing approaches where information is derived from a consensus signal from a large number of molecules attached to defined areas of the support (see, for example, Automation Technologies for Genome Characterisation, Wiley-Interscience (1997), ed. T. J. Beugelsdiijk, Chapter 10:205-225).

Generating a large ordered array of single molecules is not essential nor is it practical for approaches involving single molecule detection. This is partly because detecting events at the single molecule level requires that the molecules should be distributed on a solid support with sufficient separation between the molecules to enable each molecule to be individually resolved e.g. by optical microscopy.

In addition, and in contrast to arrays used in conventional assays where the spatial distribution of samples is essential to track the identity and data for each sample, single molecule detection allows each individual molecule to be identified and its position on a solid support to be determined. Thus, the location of each molecule can be determined without reference to a position, making an ordered array of single molecules unnecessary.

WO 00/06770 describes immobilising a mixture of molecules to a solid surface in such a way that sufficient separation between the molecules is achieved and thus to allow optical resolution at the single molecule level. In the method described therein, immobilisation is via microspheres which are bound to the solid surface. The microspheres are diluted before deposition on the solid surface to give a density of one microsphere per 100 square microns prior to attachment of the nucleic acid molecule of interest onto the microspheres. However, for this method to be effective, it is desirable to achieve attachment of only one molecule per microsphere by further dilution of the mixture of molecules prior to attachment. Moreover, as the microspheres were found to have some residual fluorescence, an additional preparation step of photobleaching the microspheres is required.

U.S. Pat. No. 6,258,454 describes a means for altering surface energy and providing functional groups on the surface. The aim is therefore to provide gross modification of the surface by mixing silane molecules with hydrophobic and hydrophilic properties. The surfaces described in the patent are unsuitable for the sparse distribution of single molecules as a prelude to their analysis because the surface densities of functional groups would be too high. At such high densities, the hydrophobicity of the surface would prevent sufficient wetting to enable the attachment of molecules or their subsequent modification in aqueous environments.

U.S. Pat. No. 5,728,203 describes the preparation of a composition comprising two or more silanes and phosphoric acid for the treatment of metal surfaces to render them coatable with paints and varnishes, or other similar treatments. The description is that of an aqueous solution of hydrolysed silanes. The selection criteria for the silanes is based upon the ability to co-polymerise and thus provide a protective coating. The combination of silanes disclosed in this document would not permit the attachment of single molecules with optically resolvable separation between them.

U.S. Pat. No. 5,866,262 describes scratch resistant coatings for spectacles. The composition is that of multiple reactive silanes that co-polymerise to provide a hardened coating on glass to prevent physical damage. The coatings generated are designed to be passive and result from the mixing of bulk quantities of silane with a polyfunctional resin modifier.

A means of local functionalisation of a modified glass surface for the attachment of molecules and their subsequent modification and analysis is disclosed in U.S. Pat. No. 5,474,796. The method describes generating features by creating chemical masks that are at least 0.1 um in diameter. The chemical groups within a feature would be entirely reactive or passive. In contrast, the feature dimensions of the present invention are generated by the controlled mixing of passive and active groups to provide features that comprise one reactive molecule per 0.1 um diameter, or preferably 1 um diameter which could not be achieved by the method described in U.S. Pat. No. 5,474,796.

U.S. Pat. No. 5,137,765 specifically describes a support comprising a mixture of a free acid group and a quaternary ammonium group. A means for achieving this is by mixing silanes. The mixtures are prepared to alter the bulk properties of the coated surfaces to create mixed ion bed resins that support the stable and quantitative attachment of proteins.

Other methods of obtaining arrays useful in the detection of single molecules would include dispensing small volumes of a sample containing a mixture of molecules onto a suitably prepared solid surface, or applying a dilute solution of the sample to the solid surface to generate a random array. However, both these methods have disadvantages: dispensing small volumes requires specialised apparatus whereas dilution of a sample is empirical and depends on quantifying and diluting each sample to be analysed. Moreover, molecules, particularly proteins, in a random dilution are likely to interact with one another thus increasing the likelihood of clustering of molecules at particular sites.

It is therefore an object of this invention to provide an improved method that permits an essentially random distribution of biomolecules on a solid surface whilst allowing a degree of control upon the density of molecules obtained.

Accordingly, in a first aspect of the invention there is provided a composition for coating a solid support to provide a sparse distribution of reactive groups in a background of passive groups comprising molecules of Formula I and Formula II which are defined as follows:

Y-X-Z-R1 Formula I

Y′-X′-Z′-R2 Formula II

and wherein

R1 is a reactive group which can form a covalent bond with a biomolecule or a group capable of forming a reactive group;

R2 is different to R1 and is present in at least a 10⁴ fold molar excess to R1;

Y and Y′ are groups which can bind to a solid surface;

X and X′ are atoms which are, at least, bivalent; and

Z and Z′ are linker groups.

A reactive group is herein defined as a functional group which is capable of reacting with another selected chemical group to form a covalent bond or a new species under specified conditions. In contrast, a passive group is defined as a chemical moiety that is not capable of reacting with the same selected group under the same specified conditions.

By sparse distribution of reactive groups is meant a distribution on the surface of the solid support of molecules of Formula I at a density of one molecule per 0.1-100 square microns, preferably, one molecule per 0.1-10 square microns, and, most preferably, one molecule per 1-10 square microns.

R1 is a reactive group which can form a covalent bond with a biomolecule or a group which can form such a reactive group. Suitable biomolecules include nucleotides and proteins. The term “nucleotide” is used to include natural nucleotides and nucleotide analogues, or a polynucleotide, which term is used to include oligonucleotides of natural or synthetic origin and which may contain nucleotide analogue residues. Polynucleotides may be single-stranded or double-stranded and may be RNA, DNA, PNA or nucleic acid mimics. Suitably, DNA may be cDNA, DNA of genomic or other origin, PCR fragments and may-include nucleotide analogue residues. Although the length of the polynucleotide is immaterial, the invention is likely to be of particular interest for the immobilisation of oligonucleotides and of PCR fragments. The term “protein” includes polypeptides, such as cytokines, receptors, antibodies and their fragments (including Fc and Fab′ fragments), other peptide fragments and amino acids that may be naturally derived or synthetic.

In a preferred embodiment the biomolecule is a nucleic acid. Direct binding of a nucleic acid to a surface via a Si-containing molecule has been described, for example, in Kumar et al. Nucleic Acids Research, 28 (14), e71(i-vi), 2000. A reactive group is a group which can form a covalent bond with a biomolecule. Suitable groups are described, for example, in Lyubchenko et. al (1992) J. Biomol. Struct & Dynamics vol 10(3)589-606, Beier M & Hoseisel J (1999) Nucleic Acids Res. Vol 27(9) p1970-1977, Joos et al. (1997) Anal. Biochem. 247 p96-101, Rogers et al. (1999) Anal. Biochem vol 266(1) p23-30 and Weetall H H (1993) Appl Biochem Biotechnol. vol 41(3) pi57-188. Accordingly, in a preferred embodiment of the first aspect, R1 is selected from —SH, —NH₂, —CN, —F, —Cl, —Br and —I.

In another embodiment, R1 is a group capable of forming a reactive group when reacted with a suitable agent. The reactive group formed is one which is capable of forming a covalent bond with a biomolecule such as a protein or nucleic acid.

For example, where R1 is a thiol group it can be reacted with a di-(organic) disulphide, where one or both of the organic groups is a leaving group to give the required functionalised surface. In another example, where R1 is an amino group it can be reacted with, for example, 3,3′-dithiopropionic acid my the presence of a coupling reagent such as 1-(3-Dimethylaminopropyl)-3-ethylcarbodiimide (EDC) or O-Benzotriazol-1-yl-N,N,N′,N′-tetramethyluronium hexafluorophosphate (HBTU) followed by reduction with dithiothreitol and reaction with a di-(organic)disulphide to yield the required functionalised surface. Alternatively, where R1 is an amino group, it may also be reacted with 2-carboxyethyl-2′-(leaving group)disulphide in the presence of a coupling reagent such as EDC or HBTU.

Disulphide bonds are very widely used to bind proteins and other biomolecules covalently onto solid surfaces. In a particularly preferred embodiment, R1 is a group of the formula —S—S-L, where L is a leaving group, i.e. a group which is readily replaced by suitably modified polypeptide or polynucleotide, such as H.

In another embodiment, reactive group R1, such as SH, or NH₂, may be reversibly protected to reduce reaction between mixed silanes during the coating of a solid support. Where R1 is SH, this can be achieved by disulphide bond formation with reactive agents such as dipyridyldisulphide. Where R1 is NH₂, this can be achieved by reaction with an amino protective group such as a t-butyloxycarbonyl group or other reagents commonly used to protect primary amines. Once a solid surface is coated with the composition, the groups protecting the reactive groups can be removed using conventional chemistry to render R1 capable of forming a covalent bond with a biomolecule such as a protein or nucleic acid.

In a preferred embodiment, the biomolecule itself can be modified so as to bind R1.

R2 is different to R1. Thus, when R1 is a reactive group, R2 is not; when R1 is capable of being activated to form a reactive group, R2 will not be activated under the same conditions.

Accordingly, in a particularly preferred embodiment, R2 is a group which will remain unreactive when treated with an agent that activates R1. Preferably, R2 is selected from —OH, —Me, —OMe, —Phe, —F, —Cl, —SO₃ and —CO₂.

Suitably, R2 is —OH or another group that will allow the surface of the slide to be substantially hydrophilic. This will create an environment which favours the attachment of the molecules such as polynucleotides or polypeptides to the reactive group, R1, on the attachment molecules of Formula I. Other “less hydrophilic” groups, such as methyl etc., may create hydrophobic pockets and thus reduce the ability of neighbouring reactive groups, R1, to interact with the molecules to be attached. Y and Y′ are the same or different. In a particularly preferred embodiment, Y and Y′ are the same. In one embodiment, Y is selected from methoxy, ethoxy and carboxy.

X and X′ are the same or different. In a particularly preferred embodiment, X and X′ are the same. In another preferred embodiment, X and/or X′ is Si. In another preferred embodiment where Y is Si, X is a polymer such as Polyethylene Glycol (PEG) or a polysaccharide such as dextran.

In a particularly preferred embodiment, where X is Si, Y is ethoxy.

Z and Z′ are the same or different. In a particularly preferred embodiment, Z and Z′ are the same. In another embodiment of the first aspect, Z and Z′ are linker groups of at least one atom up to a length determined by the size of the polymer, such as PEG or dextran. Preferably Z or Z′ are less than 10⁶ atoms, more preferably less than 100,000 atoms, more preferably less than 10,000 atoms, more preferably less than 1,000 atoms and most preferably less than 100 atoms selected from C, O, N, P, S and Si. The nature and existence of such linkers is well known in the art and is not material to the present invention. In a particularly preferred embodiment, Z is 1, 2 or 3 carbon atoms.

Suitably, Z and Z′ comprise a hydrophilic polymer. Suitably, Z and Z′ comprise a carbohydrate of at least two monmeric units or a derivative thereof. Preferably, Z and Z′ comprise a dextran or a derivative thereof. More preferably, Z and Z′ comprise cellulose or a derivative thereof. More preferably, Z and Z′ comprise polyethylene glycol (PEG) or a derivative thereof.

In a preferred embodiment, the composition comprises molecules of Formula I which are silanes. Suitable silanes are available, for example, from Fluorochem Ltd., UK. In a particularly preferred embodiment the composition comprises molecules of Formula I selected from 3-aminopropyldimethoxysilane, (3-mercaptopropyl)trimethoxysilane, (3-aminopropyl)dimethlyethoxysilane, (3-mercaptopropyl)dimethoxysilane, (4-aminophenyl)trimethoxysilane, m-amino-phenyltrimethoxysilane and (3-glycidoxypropyl)trimethoxysilane, (3-aminopropyl)methyldiethoxysilane, (3-aminopropyl)triethoxysilane, (3-aminopropyl)trimethoxysilane, (3-chloropropyl)dimethoxymethylsilane, (3-chloropropyl)triethoxysilane, (3-cyanopropyl)triethoxysilane, (3-cyanopropyl)methyldimethoxysilane, (3-glycidoxypropyl)dimethylmethoxysilane, (3-glycidoxypropyl)methyldiethoxysilane, (3-glycidoxypropyl)triethoxysilane, (3-glycidoxypropyl)methyldimethoxysilane, (3-mercaptopropyl)methyldimethoxysilane, (3-mercaptopropyl)triethoxysilane and (3-mercaptoethyl)trimethoxysilane.

Suitable molecules of Formula II are those which are able to bind to the solid support but are unable to attach biomolecules, even after treatment with the agent which can activate R1 in Formula I where R1 is an activatable group. Thus, no biomolecules will be attached to the solid support in the regions where the molecules of Formula II are bound.

In a preferred embodiment, the composition comprises molecules of Formula II which are silanes. In a particularly preferred embodiment, the composition comprises molecules of Formula II selected from [2-bis(hyrdroxyethyl)-3aminopropyl]trimethoxysilane, (4-hydroxyphenyl)trimethoxysilane, (3-hydroxypropyl)-trimethoxysilane, propyldimethoxysilane, (3-glycodoxypropyl)trimethoxysilane, (3-hydroxypropyl)methyldimethoxysilane, (4-hydroxyphenyl)trimethoxysilane, (4-hydroxyphenyl)methyldimethoxysilane, phenyltrimethoxysilane, phenyldimethylethoxysilane, propylmethyldimethoxysilane, m-aminophenyltrimethoxysilane, 4-aminophenyltrimethoxysilane, (3-aminopropyl)dimethylethoxysilane, (3-aminopropyl)methyldiethoxysilane, (3-aminopropyl)triethoxysilane, (3-aminopropyl)trimethoxysilane, (3-chloropropyl)dimethoxymethylsilane, (3-chloropropyl)triethoxysilane, (3-cyanopropyl)triethoxysilane, (3-cyanopropyl)methyldimethoxysilane, (3-glycidoxypropyl)dimethylmethoxysilane, (3-glycidoxypropyl)methyldiethoxysilane, (3-glycidoxypropyl)triethoxysilane, (3-glycidoxypropyl)methyldimethoxysilane, (3-mercaptopropyl)methyldimethoxysilane, (3-mercaptopropyl)triethoxysilane and (3-mercaptoethyl)trimethoxysilane.

In another embodiment, the composition comprising a mixture of compounds of Formula I and Formula II can form a monolayer on a solid surface.

Suitably the “attachment” molecules of Formula I and the “non-reactive” molecules of Formula II are of essentially similar structure, have substantially similar properties and differ only in the ability or lack of ability to form attachments with biomolecules. In a particularly preferred embodiment, the molecules of Formula I and Formula II are of substantially uniform size. In another embodiment, the two sets of molecules will bind to a solid support with an equivalent efficiency thus giving a substantially uniform layer of molecules on the solid support. In yet another embodiment, the interactions between molecules of Formula I and molecules of Formula II are substantially the same as those interactions amongst molecules of Formula I and interactions amongst molecules of Formula II such that the attachment molecules and non-reactive molecules can mix freely. These features ensure that the distribution of molecules in the layer on the solid support is highly uniform with attachment molecules distributed in a background of non-reactive molecules. This allows for the dilution of the attachment molecules of Formula I to be controlled to achieve a desired density of attachment sites on a solid support. The relative proportions of compounds of Formula I and II in the mixture will depend on the concentration of attachment sites desired.

Suitably, the molecules of Formula I and II are both silanes in which the molecules of Formula I are silanes possessing a suitable reactive group, R1, such as an amine, or sulphydryl group and molecules of Formula II are silanes lacking such a reactive group.

For example, where both the molecules of Formula I and II are silanes, the small and defined nature of these molecules ensures that the distribution of silane is highly uniform when the composition forms a layer on a solid support.

Accordingly, suitable compositions in accordance with the first aspect of the invention comprise a mixture of 3-aminopropyldimethoxysilane and [2-bis(hyrdroxyethyl)-3aminopropyl]trimethoxysilane or a mixture of (4-aminophenyl)trimethyoxysilane and (4-hydroxyphenyl)trimethoxysilane. The ratio of 3-aminopropyldimethoxysilane (i.e. attachment molecule): [2-bis(hyrdroxyethyl)-3aminopropyl]trimethoxysilane (i.e. non-attachment molecule) can be varied to control the density of attachment sites when the composition is attached to a solid support.

In a preferred embodiment, X and X′ are Si, Z and Z′ are polyethylene glycol, R1 is —SH and R2 is —OMe.

In another embodiment, X and X′ are Si, Z and Z′ are dextran, R1 is either —SH or —NH₂, and R2 is selected from —OH, —SO₃ and —CO₂.

In second aspect, there is provided a solid support having on its surface a composition in accordance with the first aspect of the invention.

In one embodiment of the second aspect, the surface of the support has a group, or can be modified to have a group which binds to Y. In a particularly preferred embodiment, the solid support will have surface hydroxyl groups, or can be modified to contain OH groups, which can be reacted with molecules in accordance with Formula I and II. Suitably where the surface has OH groups, Y is selected from methoxy, ethoxy and carboxy.

The solid support may be massive, e.g. a surface of a reaction vessel or the wells of a microtitre plate, or may be particulate. Of particular interest are flat surfaces which may be porous or non-porous. The material of the support should be stable against oxidation or hydrolysis, and may be inorganic e.g. silicon or titanium dioxide or aluminium hydroxide or, preferably, glass; or organic e.g. polystyrene, cellulose, polyamide and others.

In a particularly preferred embodiment, the solid support is glass or silica.

In another aspect, there is provided a solid support having on its surface a layer of attachment molecules characterised in that the attachment molecules are sparsely interspersed with non-reactive molecules. Suitably, the attachment molecules are molecules of Formula I and the non-attachment molecules are molecules of Formula II.

Preferably, the solid support will have molecules of Formula I distributed on the surface at a density of one molecule per 0.1-100 square microns, preferably, one molecule per 0.1-10 square microns, and, most preferably, one molecule per 1-10 square microns.

In another embodiment, the solid support will further comprise a biomolecule attached to R1.

In third aspect of the invention, there is provided a method for preparing a coated solid support comprising forming a composition in accordance with any embodiment of the first aspect by diluting molecules of Formula I with molecules of Formula II, incubating a solid support with said mixture and drying the solid support.

Preferably, the molecules of Formula I are diluted with molecules of Formula II at a ratio suitable for achieving a density on the solid support of one molecule of Formula I per 0.1-100 square microns preferably, one molecule per 0.1-10 square microns and, most preferably, one molecule of Formula I per 1-10 square microns.

The density of attachment of silanes in a monolayer is either known or can be calculated as described, for example, in Kallury et al. (1994) Langmuir vol.10, 492-499 and Moon et al. (1996) Langmuir vol 12, 4621-24. Examples of the monolayer density, when attached to glass, of some silanes comprising reactive groups for the attachment of biomolecules are as follows:

| Silane | Surface Coverage (molecules/nm²) |
|---|---|
| (3-aminopropyl)dimethoxysilane | 5.7^{a} |
| (3-aminopropyl)triethoxysilane | 2.4^{a} |
| (4-aminophenyl)trimethoxysilane | 2.5^{b} |

^{a}= Kallury et al. (1994) Langmuir vol. 10, 492-499,
^{b}= Moon et al. (1996) Langmuir vol 12, 4621-24.

Thus, for example, to achieve a density of one attachment molecule of (4-aminophenyl)trimethoyxsilane per μm² would require its dilution with a non-reactive molecule (4-hydroxyphenyl)trimethoxysilane at a ratio of 1:6.25×10⁶. Similar calculations can be performed to achieve any desired density for a silane coating. Such dilutions are effected prior to coating the solid support with the mixture of silanes. Ratios may also be determined empirically on an experimental basis.

Coating a solid support with a mixture of silanes (or “silanisation”) can be performed in either the vapour phase or liquid phase (see, for example, Lyubchenko et. al (1992) J. Biomol. Struct & Dynamics vol 10(3)589-606).

Preferably, a silane mixture may be applied in the liquid phase as this would permit a more uniform solution of the silanes to be contacted to the solid surface, thus maximising an even distribution of the reactive silanes (i.e. attachment molecules) in an inert background of non-reactive silanes.

Previous methods for achieving a particular density of biomolecules on a solid surface required a concentration determination of the biomolecules and dilution prior to binding the biomolecules to the support. In a solid support in accordance with the second aspect of the invention, the density of attachment molecules of Formula I and, therefore, binding sites determines the distribution and density of attached biomolecules. Accordingly, the biomolecules can be added in excess to the solid support obviating the need for accurate pre-dilution. Attachment of the biomolecules to the surface of the solid support will be possible at defined positions (i.e. where attachment molecules carrying reactive groups are present) and thus at a density which has been predetermined. The stochastic nature of binding events and the number of such reactions will ensure that an appropriate representation of the population of biomolecules binds to the surface.

In a fourth aspect of the invention, there is provided a method of immobilising a biomolecule on a solid support, which method comprises: preparing a composition in accordance with the first aspect of the invention; coating said composition onto a solid support; and providing a biomolecule comprising a group which reacts with R1 under conditions for said reaction to occur.

The biomolecule to be immobilised may be modified by being provided with a group which can interact with R1 after it has been functionalised. Where the biomolecule is a nucleic acid molecule and R1 is a thiol group, this may be done by replacing a 5′-terminal or 3′-terminal phosphate group —PO₄H with a phosphorothioate group —PO₃SH. The modified nucleotide or polynucleotide is contacted with the functionalised surface of the solid support under conditions to couple the two together by means of a sulphide exchange reaction.

Suitably, the biomolecule may be immobilised by a single bond or by a plurality of such bonds. The bonds are, preferably, stable to the conditions that may be encountered during analysis of the biomolecule e.g. conditions encountered during nucleic acid hybridisation or other procedures.

### SPECIFIC DESCRIPTION

For the purposes of clarity, certain embodiments of the present invention will now be described by way of example.

FIG. 1a is a diagram showing an example of surface bound silane binding to an oligonucleotide.

FIG. 1b is a diagram showing examples of surface bound silanes.

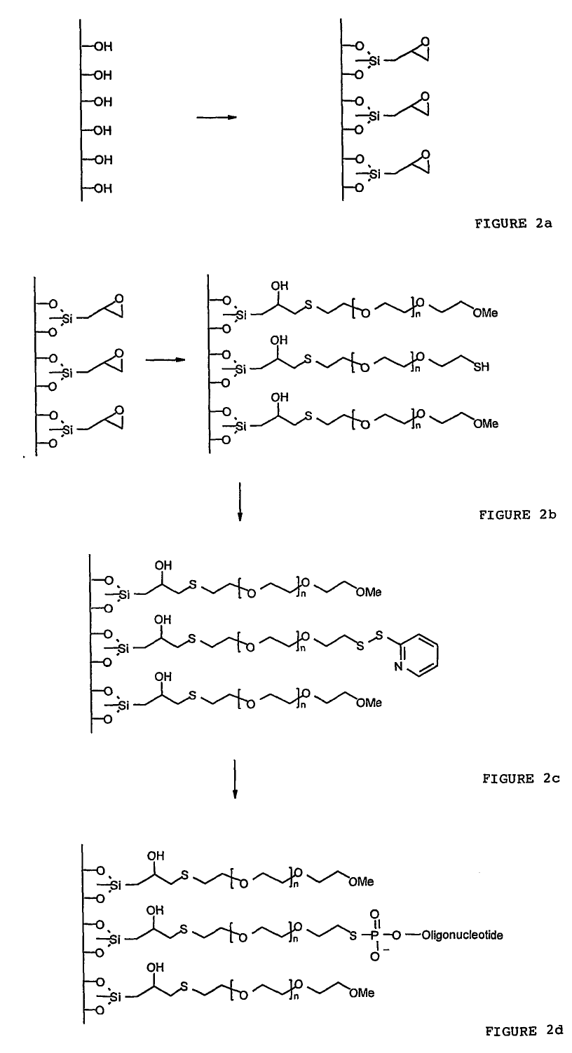

FIG. 2a-d illustrate the immobilastion of Phosphorothioate Oligonucletiotides to surfaces grafted with sparsely distributed reactive groups.

FIG. 2a is a diagram of a slide surface following silanisation.

FIG. 2b illustrates derivitisation of the silyl groups with HS-PEG-SH/HS-PEG-OCH₃.

FIG. 2c shows the slide following treatment with aldrithiol.

FIG. 2d depicts immobilisation of the phosphorothioate oligonucleotide to the slide.

### EXAMPLE 1

A silane mixture is prepared. To achieve a density of 1 molecule of (4-aminophenyl)trimothyxsilane per μm² it is diluted in (4-phenyl)trimethoxysilane at a ratio of 1:6.25×10⁶. (This dilution is based on the monolayer density for this undiluted (4-aminophenyl)trimothyxsilane being 2.5 molecules per nm²).

Silanisation in the liquid phase is carried out as follows: 3 ml of the silane mixture are added to 300 ml of dry toluene. Slides are cleaned with detergent before being baked at 125° C. to 130° C. for 1½ hours to completely remove traces of water before being soaked in the silane/toluene solution for 1 to 2 hours. Slides are then washed twice in dry toluene, followed by ethanol and dried at 100° C. for 1 hour and 60° C. for more than 10 hours. The coated slides are stored in a vacuum dessicator.

To prepare the coated slides for oligonucleotide attachment, the terminal amino groups of the silanes are reacted with 1,4-phenylene di-isothiocyanate (PDC) to convert the amino groups to amino-reactive phenylene isothiocyanate groups. The slides are soaked in a solution of 2 g/l PDC in DMF/dry pyridine (9:1 v/v) overnight. The slides are then washed in DMF, followed by ethanol and dried at 110° C. in the oven.

For binding to the slides, oligonucleotides are synthesised bearing a 5′ terminal amino group. Each oligonucleotide at a concentration of 10 μm is mixed with an equal volume of 0.1M carbonate buffer pH9 and ethylene glycol and two volumes of distilled water. The oligonucleotides are then applied to the glass surface and allowed to react at 21° C. to 22° C. for a minimum of 4 hours. The slides are then rinsed with water treated with 17% ammonia, followed by four further washes with water and once with isopropanol before drying.

### EXAMPLE 2

Mixtures of 3-aminopropyldimethoxysilane (AMS) (Sigma, UK) and [2-bis(hyrdroxyethyl)-3aminopropyl]trimethoxysilane (HAS) (Fluka, UK) were prepared in ratios of 1:0, 1:250, 1:1000, 1:2500, 1:10,000, 1:40,000; 1:160,000, 1:1000,000, 1:40000,000 and 0:1 AMS:HMS.

Prewashed glass slides (Elan, UK) were incubated overnight with a 2.5% (v/v) solution of the silane mixture in dry toluene (Fluka, UK). Excess silane mixture was removed with the following washes: 1× toluene, 1×1:1 toluene/ethanol, 2× ethanol (Fluka, UK), 2× water. The washed slides were dried and stored in a dessicator.

Prior to oligonucleotide attachment, the silane-coated slides were incubated overnight at room temperature with 1,4-diphenylenediisothiocyanate (Fluka, UK) at 2 g/l in 9:1 dry dimethylformamide (Sigma, UK)/dry pyridine to activate the reactive groups. The slides were washed with dimethylformamide followed by ethanol then dried at 110° C.

Each activated slide was incubated with a 40 microlitre aliquot of 2 micromolar solution of the Cy3-labelled oligonucleotide NH₂-GTG TGG(Cy3)AG (Interactiva GmbH, Germany) in 50 mM phosphate buffer, pH 6.0, containing 1%(v/v) Tween-20 (Sigma, UK) under a microarray slide coverslip (APBiotech, UK) for 2 h at room temperature. The slides were then washed three times in the phosphate buffer containing 0.5% SDS, followed by three washes with water. The washes were performed at 50° C. in a sonicating water bath. The slides were allowed to dry before analysis using a microarray scanner (Molecular Dynamics).

### EXAMPLE 3

Glass mirrored microscope slides, coated with 70 nm thick SiO₂ were silanised with neat 3-(glycidoxypropyl)methyl dimethoxysilane according to the method of Piehler et. al. (described in Biosensors & Bioelectronics (2000): 15, 473-481), FIG. 2a.

A solution of HS-PEG-SH (Mw. 2000) in water was prepared by adding 1 mg of HS-PEG-SH to 1 mL of deoxygenated water (sonication, He sparge) to give 10⁻³ mg μL⁻¹ (˜5×10⁻¹⁰ mol). An aliquot of 10 μL of this solution was added to a solution of 100 mg of HS-PEG-OCH₃ in 990 μL of deoxygenated water to give a dilution of 10⁴×. Similarly, 1 mg HS-PEG-SH was dissolved in 10 mL deoxygenated water to give 10⁻⁴ mg μL⁻¹ (˜5×10⁻¹¹ mol). An aliquot of 1 μL of this solution was added to 100 mg of HS-PEG-OCH₃ (Mw. 2000) in 999 μL deoxygenated water to give a dilution of 10⁶×. The diluted HS-PEG-SH/HS-PEG-OCH₃ solutions were lyophilised to give free running off-white powders. The powders were applied to the silanised faces of the microscope slides and the slides heated to 75° C. until the PEG mixtures were molten. FIG. 2b shows the slide surface following treatment with the HS-PEG-SH/HS-PEG-OCH₃ solution.

A second silanised microscope slide was placed onto the first slide, such that the molten PEG mixture was sandwiched between the silanised faces of the slides in a thin film. The slide pairs were then heated at 75° C. for 24 hours. A further pair of slides containing a film of HS-PEG-OCH₃ alone as a ‘control’ was prepared using the same method. Slide pairs were then separated while the PEG was still molten and then allowed to cool. Excess PEG was washed off the surface of the slides by rinsing with copious amounts of pure (18 MΩ) water. Slides were then dried with a stream of dry nitrogen gas. All slides were immersed in a solution of aldrithiol in isopropanol (6.4 g L⁻¹) and allowed to soak for 24 hours at ambient temperature. The slides were then removed and rinsed three times with isopropanol and allowed to dry in a dessicator. FIG. 2c illustrates the surface of the slide following treatment with aldrithiol. All slides were mounted in the chambers of a Lucidea Automated Slide Processor (Amersham Biosciences) and exposed to a solution of Cy5-labelled mono-phosphorothioate capped oligonucleotide (5′-TA ACT CAT TAA CAG GAT-3′) in 0.8M (pH 4) citrate buffer at a concentration of 2 pmol per slide (200 μL of 0.01 pmol μL⁻¹ solution per slide). Cy5 is available from Amersham Biosciences, UK. Slides were exposed to this solution for 30 minutes at room temperature, before washing with 50 mmol KCl/10 mmol TRIS.HCl pH8/2% triton buffer. Further washes with water and then isopropanol were performed before drying the slides with air at 48° C. FIG. 2d shows the immobilised oligonucleotide on the surface of the slide.

Slides were placed on a Nikon microscope fitted with a 10× objective and a Lavision CCD camera for single molecule detection. Cy5 was excited with a Helium-Neon laser at 633 nm and images collected for 1 to 10 seconds. Multiple objective fields were observed to demonstrate consistency of data obtained from the slide. Images produced by the CCD were analysed using the software (Datavis 6.1) provided by Lavision GmbH. (Goettingen, Germany). The images were subjected to non-linear slide minimum correction with a factor of 3 and non-linear concentration by a factor of 3.

The photomicrographs (not shown), encompassing a field of view of 870 μm×660 μm, demonstrated that the dilution of 1:1000000 reactive to passive groups resulted in opitcally resolvable separation of individual attached molecules. Only debris or the occasional non-specifically adsorbed cy5 labelled molecule was observed when the slide surface was coated with the molecules containing the non-reactive groups alone.

1. A composition for coating a solid support to provide a sparse distribution of reactive groups in a background of passive groups comprising molecules of Formula I and Formula II as follows:
Y-X-Z-R1 Formula I
Y′-X′-Z′-R2 Formula II
wherein
R1 is a reactive group which can form a covalent bond with a biomolecule or a group capable of forming said reactive group;
R2 is different to R1 and is present in at least a 10⁴ fold molar excess to R1;
Y and Y′ are groups which can bind to a solid surface;
X and X′ are atoms which are, at least, bivalent; and
Z and Z′ are linker groups.

2. The composition of claim 1, wherein R1 is selected from the group consisting of —SH, —NH₂, —CN, —F, —Cl, —Br and —I.

3. (cancelled)

4. The composition of claim 1, wherein R1 can be reacted with suitable agents to form reactive groups which are capable of forming covalent bonds with a biomolecule.

5. The composition of claim 1, wherein R2 is a group which will remain unreactive when treated with an agent that activates R1.

6. The composition of claim 1, wherein R2 is selected from the group consisting of —OH, —Me, —OMe, —Phe, —F, —Cl, —SO₃ and —CO₂.

7. The composition of claim 1, wherein Y and Y′ are the same.

8. The composition of claim 1, wherein Y and/or Y′ are selected from the group consisting of methoxy, ethoxy and carboxy.

9. The composition of claim 1, wherein X and X′ are the same.

10. The composition of claim 1, wherein X and/or X′ is Si.

11. The composition of claim 1, wherein Z and Z′ are the same.

12. The composition of claim 1, wherein Z and Z′ are linker groups of at least one and up to 10⁶ atoms selected from the group consisting of C, O, N, P, S and Si.

13. The composition of claim 1, wherein Z and Z′ include a hydrophylic polymer.

14. The composition of claim 1, wherein Z and Z′ include a carbohydrate of at least two monomeric units or a derivative thereof.

15. The composition of claim 14, wherein Z and Z′ include a dextran or a derivative thereof.

16. The composition of claim 14, wherein Z and Z′ include cellulose or a derivative thereof.

17. The composition of claim 1, wherein Z and Z′ include polyethylene glycol (PEG) or a derivative thereof.

18. The composition of claim 1, wherein the molecule of Formula I is a silane, selected from the group consisting of 3-aminopropyldimethoxysilane, (3-mercaptopropyl)trimethoxysilane, (3-aminopropyl)dimethlyethoxysilane, (3-mercaptopropyl)dimethoxysilane, (4-aminophenyl)trimethoxysilane, m-amino-phenyltrimethoxysilane, (3-glycidoxypropyl)trimethoxysilane, (3-aminopropyl)methyldiethoxysilane, (3-aminopropyl)triethoxysilane,(3-aminopropyl)trimethoxysilane, (3-chloropropyl)dimethoxymethylsilane, (3-chloropropyl)triethoxysilane, (3-cyanopropyl)triethoxysilane, (3-cyanopropyl)methyldimethoxysilane, (3-glycidoxypropyl)dimethylmethoxysilane, (3-glycidoxypropyl)methyldiethoxysilane, (3-glycidoxypropyl)triethoxysilane, (3-glycidoxypropyl)methyldimethoxysilane, (3-mercaptopropyl)methyldimethoxysilane, (3-mercaptopropyl)triethoxysilane and (3-mercaptoethyl)trimethoxysilane.

19. The composition of claim 1, wherein the molecule of Formula II is a silane, selected from the group consisting of [2-bis(hyrdroxyethyl)-3aminopropyl]trimethoxysilane, (4-hydroxyphenyl)trimethoxysilane, (3-hydroxypropyl)-trimethoxysilane, propyldimethoxysilane, (3-glycodoxypropyl)trimethoxysilane, (3-hydroxypropyl)methyldimethoxysilane, (4-hydroxyphenyl)trimethoxysilane, (4-hydroxyphenyl)methyldimethoxysilane, phenyltrimethoxysilane, phenyldimethylethoxysilane, propylmethyldimethoxysilane, m-aminophenyltrimethoxysilane, 4-aminophenyltrimethoxysilane, (3-aminopropyl)dimethylethoxysilane, (3-aminopropyl)methyldiethoxysilane, (3-aminopropyl)triethoxysilane, (3-aminopropyl)trimethoxysilane, (3-chloropropyl)dimethoxymethylsilane, (3-chloropropyl)triethoxysilane, (3-cyanopropyl)triethoxysilane, (3-cyanopropyl)methyldimethoxysilane, (3-glycidoxypropyl)dimethylmethoxysilane, (3-glycidoxypropyl)methyldiethoxysilane, (3-glycidoxypropyl)triethoxysilane, (3-glycidoxypropyl)methyldimethoxysilane, (3-mercaptopropyl)methyldimethoxysilane, (3-mercaptopropyl)triethoxysilane and (3-mercaptoethyl)trimethoxysilane.

20. The composition of claim 1, wherein the molecules of Formula I and II are both silanes.

21. The composition of claim 1, including a mixture selected from the group consisting of (4-aminophenyl)trimethyoxysilane and (4-hydroxyphenyl)trimethoxysilane or 3-aminopropyldimethoxysilane and [2-bis(hyrdroxyethyl)-3aminopropyl]trimethoxysilane.

22. The composition of claim 1, wherein X and X′ are Si, Z and Z′ are polyethylene glycol, R1 is —SH and R₂ is —OMe.

23. The composition of claim 1, wherein X and X′ are Si, Z and Z′ are dextran, R1 is either —SH or —NH₂, and R2 is selected from —OH, —SO₃ and —CO₂.

24. A solid support having on its surface the composition of claim 1.

25. The solid support of claim 24, wherein the solid support is glass.

26. The solid support of claim 24, wherein molecules of Formula I are distributed on the surface at a density of one molecule per 0.1-100 square microns, preferably, one molecule per 0.1-10 square microns, and, most preferably, one molecule per 1-10 square microns.

27. The solid support of claim 24, further comprising a biomolecule attached to R1.

28. The solid support of claim 27, wherein the biomolecule is selected from the group consisting of polynucleotides, oligonucleotides, proteins and polypeptides.

29. A method for preparing a coated solid support comprising forming the composition of claim 1 by diluting molecules of Formula I with molecules of Formula II, incubating a solid support with said composition and drying the solid support.

30. A method for immobilising a biomolecule on a solid support, comprising: preparing the composition of claim 1; coating said composition onto a solid support; and providing a biomolecule comprising a group which reacts with R1 under conditions for said reaction to occur.

31. The method of claim 30, wherein the biomolecule is selected from the group consisting of polynucleotides, oligonucleotides, proteins and polypeptides.

32. The composition of claim 1, wherein said biomolecule is selected from the group consisting of oligonucleotide, polynucleotide, nucleic acid, protein and polypeptide.
